(19) United States
(12) Patent Application Publication
(10) Pub. No.: US 2012/0221256 A1
(43) Pub. Date: Aug. 30, 2012

(54) FUNCTIONAL CHARACTERIZATION OF BIOLOGICAL SAMPLES

(75) Inventors: Richard D.A. Heal, Dorchester (GB); Alan T. Parsons, Dorchester (GB); Michael J. Hudson, Buckinghamshire (GB); Scott Nicol, Iwade (GB)

(73) Assignee: Midas Mediscience, Crowthorne (GB)

(21) Appl. No.: 13/341,522

(22) Filed: Dec. 30, 2011

Related U.S. Application Data
(63) Continuation-in-part of application No. 10/551,070, filed on Sep. 27, 2005.

(30) Foreign Application Priority Data
Mar. 29, 2003 (GB) 0307352.5

(51) Int. Cl. G06F 19/00; G01N 33/94

(52) U.S. Cl. 702/21; 702/19

(57) ABSTRACT

The invention relates to systems and methods for characterizing tissue biopsies, cells and organisms as a result of predictable responses to known compounds. A sensor is used to detect plurality of features indicative of physiological activity in response to the external. A vector quantity comprising a number of dimensions equal to a number of different features is derived from the signal output of said sensor array and compared to one or more reference values to generate a physiological ‘fingerprint’.

### REFERENCE TO RELATED APPLICATIONS

This application is a continuation-in-part of U.S. application Ser. No. 10/551,070, filed Sep. 27, 2005, which is a U.S. National Stage of International Application No. PCT/GB2004/001228, filed Mar. 23, 2004, which claims priority to GB Application No. 0307352.5, filed Mar. 29, 2003. The contents of each of these applications are herein incorporated by reference in their entireties.

### TECHNICAL FIELD

The present invention relates to the characterization of biological samples, such as tissues, cells or organisms, utilized in the healthcare, pharmaceutical, cosmetic and environmental sectors for research, monitoring or commercial purposes.

### BACKGROUND

It has become the case that in the search for evermore effective pharmaceutical compounds an ever-increasing amount of time, effort and resources have been devoted to identifying and isolating potentially beneficial chemical compounds. Traditionally, the approach has been to select a molecular target within a biochemical pathway, such as an enzyme or a receptor, where interaction with the target by a compound could lead to changes which treat the disease. Typically, the interaction would take the form of the compound inhibiting or exciting the pathway. Clearly, a large number of targets will be under investigation at any one time. In order to evaluate potentially useful compounds against the target it is necessary to produce samples of the compounds for testing. The target is then screened in a series of tests against these compounds with a view to eliminating those compounds which are unsuitable and to identify those compounds that are potentially valuable. It is sometimes the case that there may be sufficient biostructural information on the molecular target to suggest the design of potentially valuable compounds. Even so, for the most part hundreds of thousands of compounds are typically screened using automated, robotic technology. Typically, the entire process from initial selection of a target through to the identification and characterization of candidate compounds can take several years.

Once identified as potentially of value in this initial screening phase, compounds showing the appropriate activity are subjected to further screens with the aim of determining their level of potency and selectivity for the target. From these data, leads will be identified.

Once a potential candidate compound has been identified, it is then subjected to further development including more screening to meet the needs of various studies both clinical and non-clinical studies. The biological effects of a compound will be assessed, wherever possible avoiding using animals in safety testing. Thus, cells in culture are an attractive alternative for the basis for such investigations. Increasingly automation is being applied to such assessment and whilst for the most part conventional assaying techniques are utilized there have been some initial attempts at employing automated techniques for cell-based assays.

One such technique which has been applied to the analysis of cell culture in response to a compound is that set out in U.S. Pat. No. 6,377,057 which describes a technique and apparatus for classifying biological agents according to the spectral density signature of evoked changes in cellular electric potential. It is suggested in the '057 patent that the approaches it teaches are intended to go beyond those previous attempts to measure cellular electric potential. Such early attempts have produced output more suited to interpretation by an experienced neuroscientist. Indeed, although such tools have been available to researchers and expert practitioners such as cardiologists since the early 1970's, it is suggested that the invention disclosed in the '057 patent is intended to be of more general use. As such the patent discloses an analysis method based on interpreting the power spectral density (PSD) of a cellular response. Thus, whilst the '057 patent teaches that the technique is capable of determining the characteristics of test compounds and identifying such previously known or unknown compounds, analysis based purely on the spectral density changes of such evoked membrane potential or action potential is considered to limit the value of the results obtained in the interests of reducing the complexity of analysis.

### SUMMARY OF THE INVENTION

The invention relates to systems and methods for characterizing and screening known or unknown compounds or agents based on the predictable, consistent response of a defined living tissue biosensor, and equally, for characterizing biological samples, such as living tissues, cells or organisms based on their electrophysiological response to various known compounds or other stimuli. The systems of the invention utilize a signal processing algorithm configured to identify and characterize electrical (biological) signals derived from electrically active cells and tissues as a result of changes in exposure to chemical compounds, or other stimuli, which may affect ion channels.

In contrast to prior techniques for screening compounds using biological measuring systems, the systems and methods described herein measure changes in a particular dimension from any given baseline resting point and only consider the amplitude and direction of the change, not the starting position. Thus, the systems and methods of the invention can be used to work from a set baseline or from a moving baseline, hence providing its own ‘internal control’ compared to many other biological measuring systems which are based on the determination of absolute measurements or amounts, not changes in direction and amplitude. This is particularly important when comparing responses of organisms or biological tissues since here the response direction is assumed to be characteristic of the functional ion channel or biological response, while the amplitude may depend on substrate availability or status of the subject.

In one aspect, the invention provides systems and methods for characterizing functional activity of an isolated biological sample by exposing the sample to an external stimulus. A plurality of features indicative of physiological activity is detected in response to the external stimulus using a sensor. The sensor can be a single sensor, such as a single electrode, or a sensor array (e.g., a microarray including a plurality of electrodes). A vector quantity comprising a number of dimensions equal to a number of different features is derived from the signal output of said sensor array. This vector quantity is compared to a baseline level of the physiological activity of the sample prior to exposure to the external stimulus to generate a physiological fingerprint of the biological sample. This fingerprint can then be compared to a reference, such as a library that includes a plurality of predetermined behavioral features (e.g., known or predicted responses to various known compounds/stimuli) of the particular biological sample. The comparison is indicative of one or more functional characteristics of the biological sample. Such method is useful for quality assurance of cells, tissues, and microorganisms.

The external stimulus can be a natural, or a synthetic stimulus. Examples of natural stimuli include but are not limited to toxins, such as crab toxin, saxitoxin, Botulinum toxin, or Tetrodotoxin; or a cell (e.g., bacterial cells such as Vibrio bacteria or a histidine-producing bacterium). Examples of synthetic stimuli include diagnostic agents, biomarkers, or chemical compounds. In one particular embodiment, the invention relates to methods for characterizing biological samples by detecting electrophysiological changes in response to cholinesterase inhibitors such as organophosphates and carbamates. Examples of organophosphates include Echothiophate, Diisopropyl fluorophosphate, Cadusafos, Cyclosarin, Dichlorvos, Dimethoate, Metrifonate (irreversible), Sarin, Soman, Tabun, VX, VE, VG, VM, Diazinon, Malathion and Parathion. Examples of carbamates include Aldicarb, Bendiocarb, Bufencarb, Carbaryl, Carbendazim, Carbetamide, Carbofuran, Carbosulfan, Chlorbufam, Chloropropham, Ethiofencarb, Formetanate, Methiocarb, Methomyl, Oxamyl, Phenmedipham, Pinmicarb, Pirimicarb, Propamocarb, Propham and Propoxur.

In some embodiments the external stimulus (e.g., cell, biomarker, diagnostic agent, or chemical compound) is known.

The stimulus may also be an environmental stimulus, such as is a change (increase or decrease) in atmospheric pressure, a change (increase or decrease) in temperature, and/or a change (increase or decrease in one or more of O₂, N₂, NO, NO₂, NO₃, NO, CO and CO₂levels.

The biological sample may be any living tissue (e.g., biopsied tissue) or a cell sample comprising one or more functional receptors such as ion channels. In certain embodiments, the biological sample is an array of different living tissues or cells that include one or more functional receptors such as ion channels. The array of different living tissues or cells can be of specific origins or selected for their sensitivity to specific compounds. Alternatively, the biological sample can be a tissue, a cell sample, or an array of different tissues or cells obtained from a cadaver. In particular embodiments, the biological sample includes one or more electrically active cells, such as primary cells derived from heart tissue, stem cells (embryonic or non-embryonic), cardiomyocytes, muscle cells, or neuronal cells. Preferably such electrically active cells are human cells.

The physiological response to the external stimuli can be static or changing physiological activity. Types of physiological activity include intracellular activity, extracellular activity, or a combination thereof. The detected physiological activity is preferably electrical. However, any chemical, fluorescent, or luminescent activity falling within the electromagnetic spectrum can also be detected using the systems and methods described herein.

In certain embodiments, the detected feature is an amplitude dependent feature, such as an intracellular or extracellular electrical signal on the external cell membrane.

The vector quantity is derived using a clustering algorithm, such as a polythetic agglomerative algorithm, a k-means algorithm or an iterative relocation algorithm.

These and other aspects of the invention are described in further detail in the figures, description, and claims that follow.

### BRIEF DESCRIPTION OF THE DRAWINGS

In order to understand more fully the invention, a number of embodiments thereof shall now be described by way of example and with reference to the accompanying drawings, in which:

FIG. 1, is a diagrammatic view of an analysis system in accordance with one aspect of the present invention;

FIG. 2, is plan view of a micro-electrode array (MEA) forming part of the system of FIG. 1.

FIG. 3, is a flow chart indicative of a method of analysis in accordance with a further aspect of the present invention;

FIG. 4 is a diagrammatic view of a sensor in accordance with a further aspect of the invention; and

FIG. 5 is a graph illustrative of a response to a compound output of the sensor of FIG. 4.

FIG. 6 is a table illustrating a feature set selected for use with the invention;

FIG. 7 is a matrix indicative of a set of responses across a feature set for example compounds in accordance with the invention;

FIG. 8 is a vector diagram based on the responses of FIG. 7.

### DETAILED DESCRIPTION

### Screening and Analysis of Compounds

In one aspect, the invention relates to systems and methods for screening/analyzing known or unknown compounds. The systems of the invention utilize a signal processing algorithm configured to identify and characterize electrical (biological) signals derived from electrically active cells and tissues in a micro-electrode array format as a result of changes in exposure to chemical compounds which may affect ion channels (including cation-channels and anion-channels). The systems and methods provided by the invention contribute to the understanding of drug properties and are commercially useful for screening and characterization of chemical compounds (known and unknown).

In contrast to prior techniques for screening compounds using biological measuring systems, the systems and methods described herein measure changes in a particular dimension from any given baseline resting point and only consider the amplitude and direction of the change, not the starting position. Thus, the systems of the invention can be used to work from a set baseline or from a moving baseline, hence providing its own ‘internal control’ compared to many other biological measuring systems which are based on the determination of absolute measurements or amounts, not changes in direction and amplitude. This is particularly important when comparing responses of organisms or biological tissues since here the response direction is assumed to be characteristic of the functional ion channel or biological response, while the amplitude may depend on substrate availability or status of the subject.

In one embodiment, the invention relates to a sensor and related systems (e.g., software systems) configured to analyze cellular and tissue responses to controlled stimuli. The sensor can be a single sensor, such as a single electrode. Alternatively, the sensor can be a sensor array, such as a micro electrode array (MEA). The software and/or firmware include a library of feature sets corresponding to experimental or otherwise derived responses to a particular compound/stimulus, to which the particular cellular network deposited on the sensor has been exposed. As such, the systems of the invention perform an identification and preferably classification function in relation to a pre-defined set of feature sets.

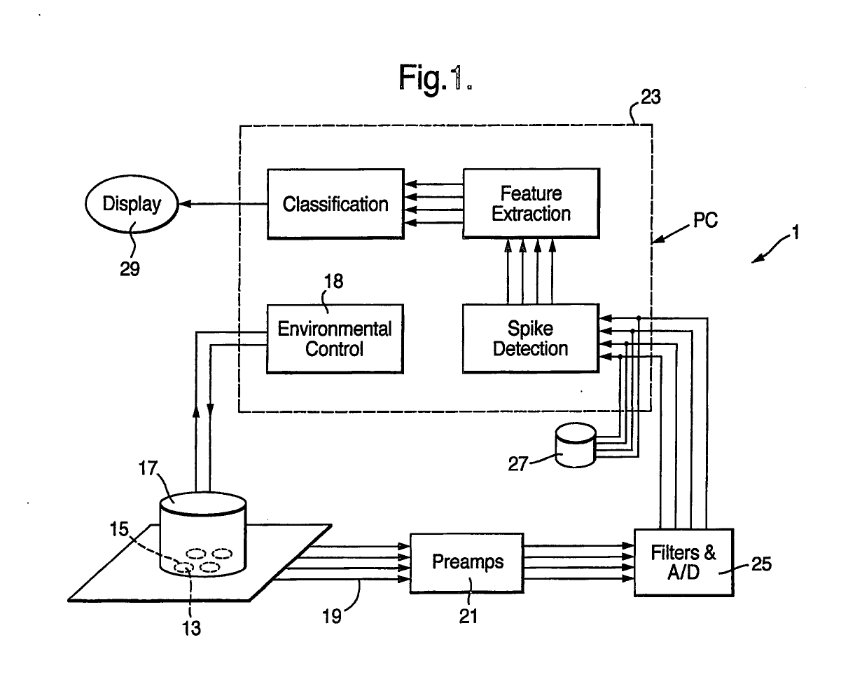

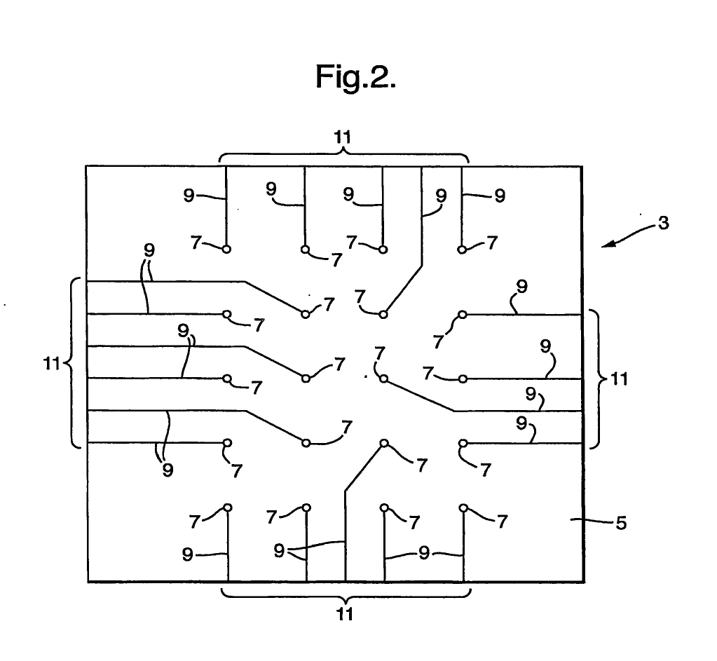

Referring now to FIG. 1, an exemplary embodiment of an analysis system 1 is shown in which an MEA 3 (see FIG. 2) provides a site onto which a cellular network is deposited. The nature of the cellular network is described in respect of a particular example below, namely a network made up of a plurality of cardio-myocytes. However, it will be appreciated from the outset by those skilled in the art that this particular example of a cellular network is purely exemplary and any reference to cellular network should not be taken to mean purely the example 5 cited herein. Any cardiomyocyte-like cells may be used with the systems described herein, including cells that express an ion channel profile typical of a ventricular or atrial cardiomyocyte cell, cells that form a functional syncitium, and/or cells that beat (contract/expand, either spontaneously or in response to a chemical or electrical stimulus) in a rhythmic fashion. Examples of such cells include, but are not limited to cardiomyocytes and primary cells derived from heart tissue. Cardio-myocyte-like cells may be derived from any species that has a heart-like organ that physically pumps fluids around their body but typically refers to vertebrate species, particularly mammals, birds, reptiles and fish, with a myogenic muscular organ that functions as part of a circulatory system to distribute blood around the body via rhythmic contraction and expansion. Examples of other suitable tissue or cells include, but are not limited to, stem cells (embryonic or non-embryonic), muscle cells, or neuronal cells Likewise, it will be appreciated from the outset by those skilled in the art that the examples of an MEA described throughout the examples herein is purely exemplary and any reference to sensors should not be taken to mean purely the sensor arrays (e.g., MEAs) described in the examples below. A single sensor (e.g., a single electrode) may be used in the systems described herein.

Referring again to FIGS. 1 and 2, the MEA 3 comprises a bio-compatible substrate 5 on which are surface mounted a plurality of electrodes 7 each of which is connected by traces 9 to an edge connector 11. The MEA 3 is insertable within a receptacle 13 formed at the bottom of a well 15. The receptacle 13 is provided with contacts for the edge connector 11. The well 15 is hermetically sealed and forms the environment or chamber 17 within which a compound is analyzed. The chamber is itself connected via a ribbon connector 19 to the input of an amplifier unit 21. The combination of the edge connector 11 and receptacle 13 provides for easy insertion and removal of MEAs 3 for analysis of different compounds. Although FIG. 2 illustrates a square array made up of equidistant electrodes, alternative array layouts are contemplated having, for example, non-uniform electrode pitch and layout. The adoption of a particular layout is predicated on the requirement that an electrode 7 should be capable of detecting electrical activity from a single cell in a network when arranged on the MEA 3. Clearly, the packing or form factor of the MEA 3 must be such that it can be correctly and easily inserted and removed from the receptacle 13 in the well 15.

Outputs from each electrode 7 passes via the aforementioned cable interconnect 19 to the amplifier unit 21 where the output is amplified. The amplifier unit 21 is a multichannel device capable of providing a gain of around 1000 to each channel. Typically, sufficient channels are available to allow each electrode 7 of the MEA 3 to be mapped to a channel. Depending on the configuration of the MEA 3 there may be need for more or less channels for satisfactory data collection. The amplifier unit 21 itself is interfaced to a PC-based data acquisition system 23. The PC system 23 incorporates an Analogue to Digital conversion card 25 coupled via a PCI bus to a central processor unit. The card 25 provides the external connections necessary to interface the analogue output of the amplifier 21 to the acquisition system 23. The card 25 is capable of sampling the amplified channel data from the amplifier unit 21 at up to 50 kHz/channel. The central processor unit carries out instructions provided by software and/or firmware necessary to analyze the digitized data. The data may be analyzed in real time as events occur on the MEA 3 or retrieved later from a storage device 27 such as a hard disk. In the former case, the storage device 27 may still be utilized to archive the data for later repeated or further analysis. The ability to proceed with real time as opposed to or off-line analysis will in some part depend on the rate at which data is generated and the storage capacity of the system 23. The nature of the cellular network placed on the MEA 3 determines the sampling rate. Thus the software and/or firmware is provided with logic to enable the system 23 to operate at an optimum sampling rate for the particular cellular network taking into account any limitations of processor speed and storage capacity. Thus, in the case of a cellular network made up of cardiomyocytes, activity may occur relatively slowly over a 100 mS window whereas in the case of neurons, activity may be present in much shorter windows of around 15 to 25 mS. In the former case, a relatively lower sampling rate may be adopted by the system 23, with the relevant control signals being provided to the Analogue to Digital Converter 25, than is needed for equivalent resolution of detail in the data derived from a neuron network. The PC system 23 is provided with a VDU 29 and printer for the presentation of results.

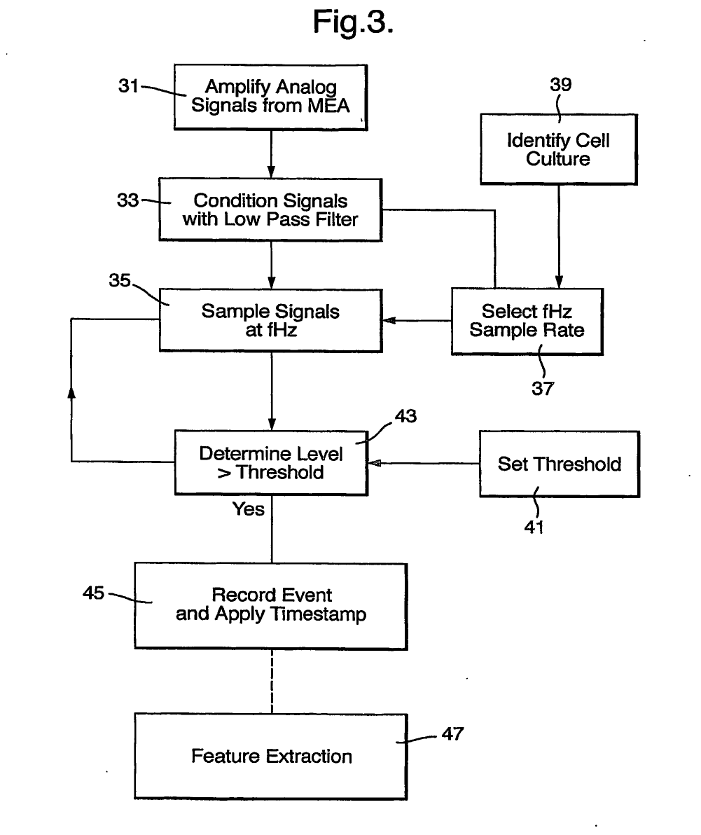

In use, and referring especially to FIG. 3 again figure of the order of 1000 is applied to each analogue channel which, in the example whose preparation is described below, namely a cardio-myocyte cellular network, has a pre-amplification value of around 100 microvolts to 2 millivolts. At this stage, the output from the electrodes 7 on the MEA 3 is an analogue signal. Clearly, before digital signal processing can be applied there is a need to digitize the signal. The rate at which the analogue signal is sampled must be selected to be high enough to ensure that all the features of interest in the electrical activity of a cellular network deposited on the MEA 3 are available to the digital signal processing unit. As a first step, the signals are amplified 31 and, the analogue signal from each electrode 7 is conditioned 33, in this case by low pass-filtering to remove unwanted high-frequency components. The filtered analogue signal on each channel is sampled 35 at a rate which may be as much as 50 kHz the actual rate 37 depending 39 on the nature of the cellular network placed on the MEA 3. In the case of a cardio-myocyte network, an effective sampling rate has been found to be 10 kHz.

By selecting a sampling rate of 10 kHz in the example of a cardiac myocyte cellular network, sufficient resolution is achieved without excessive data collection. For long-term recordings greater than 1 minute in duration, data may be stored as a series of ‘cut-outs’ of the electrical activity from the cardiac myocyte cells. Each cellular event stored as a ‘cut-out’ is determined by setting 41 a threshold level (usually a positive value) of at least 2 root-mean-square values of the noise above the baseline. For each event, a time stamp is recorded at the point at which the threshold level is crossed 43. In addition, electrode raw data 15 msec before and 85 msec after the threshold level has been crossed is stored. Data is saved to a file on the hard disk in a mcd file format (approximately 10 Mb per electrode for a 60 second recording).

The data stored on the hard disk is representative of the changes in electrical activity that occur within a cellular network and are typically in the form of action potentials or spikes. As will be described further below changes occur to the shape of the spikes and their temporal and spatial pattern when a compound is introduced to a cellular network. The electrical activity data from the cellular network is analyzed in software by imposing temporal and spatial information onto a model of the electrode array. In this way, both local and global properties of the electrical activity across the tissue sample can be identified and quantified. Examples of local properties are the peak height, amplitude or depolarization time of an action potential detected at an individual electrode. Examples of global properties are the beat frequency and propagation speed of action potentials across the culture. These various properties are referred to as features. The process of feature extraction 47 may then be performed on this digitized data.

In a variant of the present embodiment, before data is stored in a data file, the features themselves may be used to reduce the requirements for data storage. Thus the spikes can first be identified using a threshold detector, catalogued and stored, the rest of the data being ignored. Since the temporal length of a spike is typically much less than the time separation between spikes this procedure requires less storage capacity.

A non-exhaustive set of features identifiable by the analysis system 23 is listed below.

### EXAMPLES OF FEATURES

Mean Spike Rate—the number of spikes observed in a channel divided by the record length. The mean spike rate feature may be updated every minute or few minutes rather than over the whole course of an experiment.

Spike Rate Variability—calculated from the time differences between consecutive spikes, averaged over all channels.

Spike Speed—the propagation speed of the spike pulse across the MEA plate, calculated for each pulse from the spike time arrivals at each selected channel using a least mean squares fit to the data on the assumption of a single plane wave pulse propagating with constant speed.

Arrival Angle—the direction of propagation of the spike pulse.

Increase In Peak Level—the relative increase between control and test data in the maximum level of the spike profile averaged over all spikes and all selected channels.

Increase in Trough Level—the relative increase between control and test data in the minimum level of the spike profile averaged over all spikes and all selected channels.

Increase in Peak-to-Trough Level—the relative increase in the range of spike profile averaged over all spikes and all selected channels.

Increase in Absolute Peak Level—the relative increase in the maximum absolute level of the spike profile averaged over all spikes and all selected channels.

Increase in Rise Time from 10%—the relative increase in the time for a spike to achieve its maximum level starting from a level of 10%/o of the maximum, averaged over all spikes and all selected channels.

Increase In Rise Time from 20%—the relative increase in the time for a spike to achieve its maximum level starting from a level of 20% of the maximum, averaged over all spikes and all selected channels

Increase in Recovery Time to 10%—the relative increase in the time for a spike to recover to 10% of its minimum value starting from the minimum value, averaged over all spikes and all selected channels

Increase In Recovery Time to 20%—the relative increase in the time for a spike to recover to 20% of its minimum value starting from the minimum value, averaged over all spikes and all selected channels

Increase in Peak-to-Trough Time—the relative increase in the time between the maximum level and the minimum level in a spike profile, averaged over all spikes and all selected channels.

Increase In Absolute Profile Area—the relative increase in the area under the modulus profile, averaged over all spikes and all selected channels.

Increase In Profile Rise Area—the relative increase in the area under the profile between the start and the maximum value, averaged over all spikes and all selected channels.

Increase in Profile Recovery Area—the relative increase in the area under the profile between the minimum value and the end, averaged over all spikes and all selected channels.

Increase In Absolute Profile Recovery Area—the relative increase in the area under the modulus profile between the minimum value and the end, averaged over all spikes and all selected channels.

Increase in Profile Correlation Coefficient—the normalized cross-correlation between the control and test spike profiles, averaged over all spikes and all selected channels.

Increase in Profile Variance—the relative increase in the variance of the spike profile, averaged over all spikes and all selected channels.

Increase in Profile Skewness—the relative increase in the skewness of the spike profile, averaged over all spikes and all selected channels.

Increase in Profile Kurtosis—the relative increase in the kurtosis of the spike profile, averaged over all spikes and all selected channels.

Increase in maximum of wavelet transform—the relative increase in the maximum value over scale and time delay of the wavelet transform of the spike profile, using for example a Daubechies' wavelet of order 2 here and below, averaged over all spikes and all selected channels.

Increase in variance of wavelet transform—the relative increase in the variance of the wavelet transform values of the spike profile, summed over scale and time delay, averaged over all spikes and all selected channels.

Wavelet cross-correlation coefficient—the normalized cross-correlation in scale and time delay between the wavelet transforms of the control and test spike profiles, averaged over all spikes and all selected channels.

Increase in wavelet transform transfer coefficient—similar to the wavelet cross-correlation coefficient, except that it is normalized by the autocorrelation of the wavelet transform of the control data, instead of by the square root of the product the autocorrelation of the wavelet transform of the control data and the autocorrelation of the wavelet transform of the test data.

Increase in profile entropy—the relative increase in the entropy of the spike profile as determined from its histogram, averaged over all spikes and all selected channels.

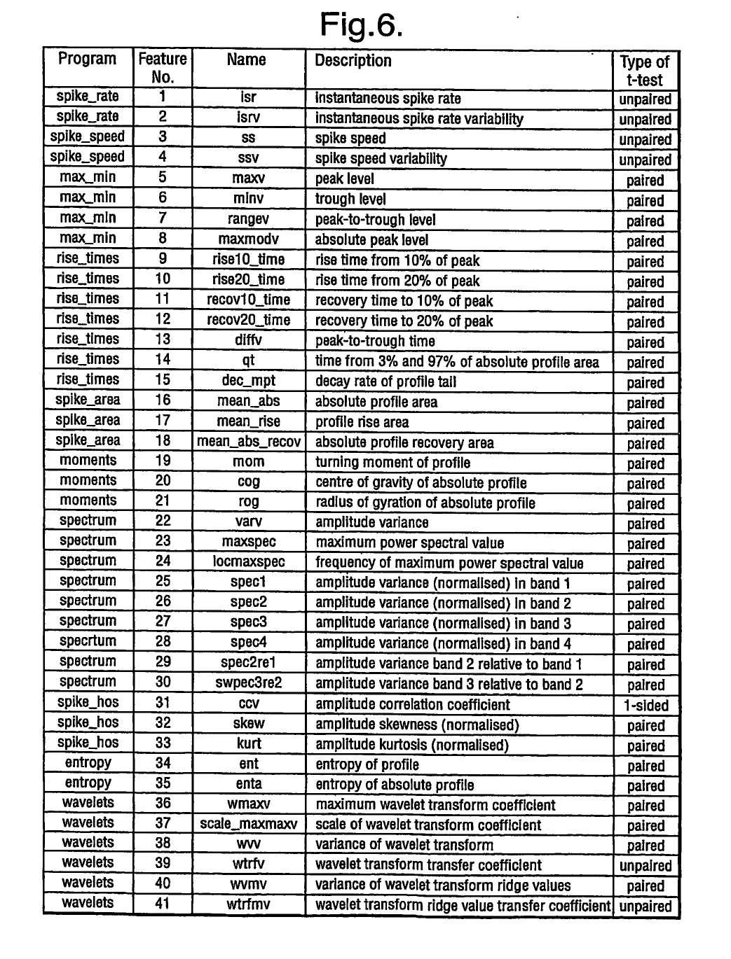

Another feature set which is believed to particularly effective in forming the basis for analysis is set out below and repeated in tabular form as FIG. 6 of the drawings. This feature set provides a numerical description of the various changes in the heart beat profile when a drug is applied

Instantaneous Spike Rate—the relative increase between control and test data in the instantaneous spike rate averaged over all selected channels.

Instantaneous Spike Rate Variability—the relative increase between control and test data in the temporal variability of the instantaneous spike rate.

Spike Speed—the relative increase between control and test data in propagation speed of the spike pulse across the MEA plate, calculated for each pulse from the spike time arrivals recorded at each selected each channel.

Spike Speed Variability—the relative increase between control and test data in the temporal variability of the spike speed.

Peak Level—the relative increase between control and test data in the maximum value in the averaged spike profile obtained by averaging the profiles of all the spikes in each selected channel.

Trough Level—the relative increase between control and test data in the minimum value in the averaged spike profile obtained by averaging the profiles of all the spikes in each selected channel.

Peak-to-Trough Level—the relative increase between control and test data in difference between the maximum and minimum values in the averaged spike profile obtained by averaging the profiles of all the spikes in each selected channel.

Absolute Peak Level—the relative increase between control and test data in the maximum value in the absolute averaged spike profile obtained by averaging the profiles of all the spikes in each selected channel.

Rise Time to 10%—the increase between control and test data in the time for an averaged spike to achieve its maximum level starting from a level of 10% of the maximum.

Rise Time to 20%—the increase between control and test data in the time for an averaged spike to achieve its maximum level starting from a level of 20% of the maximum.

Recovery Time to 10%—the increase between control and test data in the time for an averaged spike to recover to 10% of its minimum level.

Recovery Time to 20%—the increase between control and test data in the time for an averaged spike to recover to 20% of its minimum level.

Peak-to-Trough Time—the increase between control and test data in the time between the maximum level and the minimum level in the averaged spike profile.

QT Time—the increase between control and test data in the time between the 3% and 97% cumulative points of the absolute area under the averaged spike profile.

Profile decay rate—the increase between control and test data in the decay rate of the tail of the averaged profile.

Absolute Profile Area—the relative increase between control and test data in the area under the modulus of the averaged profile.

Profile Rise Area—the relative increase between control and test data in the area under the averaged profile between the start and the maximum value.

Absolute Profile Recovery Area—the relative increase between control and test data in the area under the modulus of the averaged profile between the minimum value and the end.

Profile turning moment—the relative increase between control and test data of the temporal turning moment of the averaged profile.

Absolute profile centre of gravity—the relative increase between control and test data of the centre of gravity of the absolute averaged profile.

Absolute profile radius of gyration—the relative increase between control and test data of the radius of gyration of the absolute averaged profile measured about its centre of gravity.

Amplitude Variance—the relative increase between control and test data in the variance of the averaged spike profile.

Maximum spectral value—the relative increase between control and test data in the maximum value of the power spectrum of the averaged spike profile.

Frequency of maximum spectral value—the relative increase between control and test data in the frequency of the maximum value of the power spectrum of the averaged spike profile.

Amplitude Variance in Frequency Band 1—the relative increase between control and test data in the variance of the averaged spike profile, normalized by the total variance, in the frequency band 0-250 Hz.

Amplitude Variance in Frequency Band 2—the relative increase between control and test data in the variance of the averaged spike profile, normalized by the total variance, in the frequency band 250-500 Hz.

Amplitude Variance in Frequency Band 3—the relative increase between control and test data in the variance of the averaged spike profile, normalized by the total variance, in the frequency band 500-750 Hz.

Amplitude Variance in Frequency Band 4—the relative increase between control and test data in the variance of the averaged spike profile, normalized by the total variance, in the frequency band 750-1000 Hz.

Amplitude Variance in Band 2/Band 1—the relative increase between control and test data in the ratio of the variances in bands 2 and 1 in the spectrum of the averaged spike profile.

Amplitude Variance in Band 3/Band 2—the relative increase between control and test data in the ratio of the variances in bands 3 and 2 in the spectrum of the averaged spike profile.

Amplitude Correlation Coefficient—the normalized cross-correlation between the averaged control and averaged test spike profiles.

Amplitude Skewness (normalized)—the relative increase between control and test data in the skewness, normalized with respect to the total variance, of the averaged spike profile.

Amplitude Kurtosis (normalized)—the relative increase between control and test data in the kurtosis, normalized with respect to the total variance, of the averaged spike profile.

Entropy of profile—the relative increase between control and test data in the entropy of the averaged spike profile as determined from its histogram.

Entropy of absolute profile—the relative increase between control and test data in the entropy of the absolute averaged spike profile as determined from its histogram.

Maximum wavelet transform coefficient—the relative increase between control and test data in the maximum value over scale and time delay of the wavelet transform of the averaged spike profile, using a Daubechies wavelet of order 2 here and below.

Scale change of wavelet transform coefficient—the relative increase between control and test data in the scale of maximum value over scale and time delay of the wavelet transform of the averaged spike profile, using a Daubechies wavelet of order 2 here and below.

Variance of wavelet transform—the relative increase between control and test data in the variance of the wavelet transform values of the averaged spike profile, summed over scale and time delay.

Wavelet transform transfer coefficient—wavelet cross-correlation coefficient normalized by the autocorrelation of the wavelet transform of the control data.

Variance of wavelet transform ridge values—the relative increase between control and test data in the variance of the vector of wavelet transform values of the averaged spike profile obtained by taking the maximum value at each scale.

Wavelet transform transfer coefficient ridge values—wavelet cross-correlation coefficient of the maximum vector as defined above, normalized by the autocorrelation of the corresponding vector in the control data.

It should be noted that not all the above features are amplitude dependant. Thus, features which depend on the recovery rate of the cellular network may be used to assist in detection and classification. Furthermore, although the above features may or may not be present to a greater or lesser extent in the electrical activity of the cellular network it is considered that similar features may be identified from the chemical behavior of the network in respect of its fluorescent and/or luminescent activity.

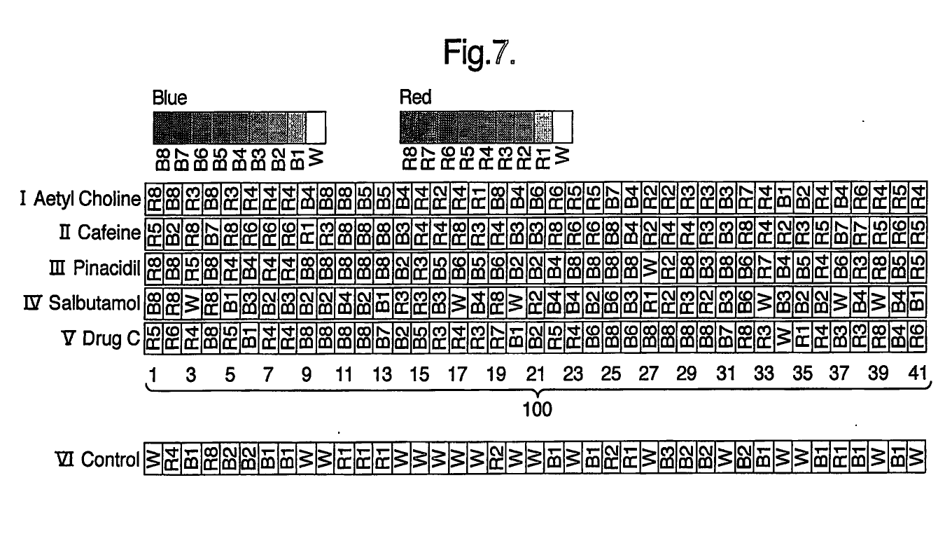

FIG. 7, exemplifies the results of a feature set analysis in a matrix format for a set of different compounds acetylcholine I, caffeine II, pinacidil III, salubutamol IV and drug C V, such as might result from carrying out activities set out in the examples below. The numerals 100 beneath the columns are indicative of respective features in a feature set and the level of shading of the boxes indicates the nature of the response. Results from a control VI are shown separately.

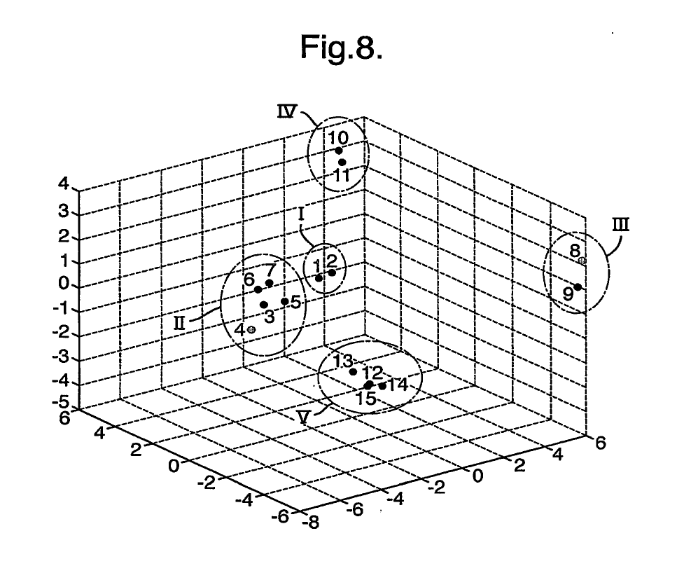

FIG. 8 shows the outcome of plotting the results of a feature set as a vector quantity in three-dimensional space for the compounds of FIG. 7 where clustering of the results is evident for each of the compounds identified by their respective reference numerals.

The sequence of activities necessary to analyze a compound is set out below. These activities are a combination of physical processes taken in relation to the deposition of a cellular network on the MEA and the compound to be tested together with signal processing activity which is carried out utilizing the PC-based system 23. Firstly a cellular network is cultured and deposited on the MEA 3. An example of the steps required in this regard using cardio-myocyte cells is as follows:

(a) Heart tissue is isolated from rat embryos (E15-E18) or neonates.

(b) The heart tissue is minced using a scalpel and placed into cold (4° C.) Ca^{2+/}Mg²⁺—free Hanks balanced salt solution (HBSS).

(c) The tissue is washed (3 times) in fresh HBSS and replaced with 0.05% trypsin in HBSS.

(d) Incubate 10 min. at 37° C. and discard supernatant.

(e) Add fresh DNase type II solution (10,000 Units/ml) for 2 min.

(f) Add fresh trypsin and incubate at 37° C. for 8 min.

(g) Remove supernatant and place into HAMS F10 containing 36% FCS, 0.5% insulin/transferrin/selenite, 6 mM L-glutamine and 2% penicillin/streptomycin (200 mM stock).

(h) Collect cells from suspension (1500 rpm, 5 mins) and resuspend in HAMS F10 containing 10% FCS, 0.5% insulin/transferrin/selenite, 6 mM L-glutamine and 2% penicillin/streptomycin (200 mM stock).

(i) Repeat steps (e)-(h) 5-8 times.

(j) Perform differential adhesion by incubating pooled cell suspensions in a treated tissue culture flask for 2 hrs at 37° C.

(k) The final cell suspension is counted and seeded onto fibronectin treated MEA plates at 50K per plate in a 100 μl volume.

The cell suspension is deposited on the MEA 3 such that each electrode 7 is in contact with a respective cell.

Once the cellular network is in place on the MEA 3, the MEA 3 is inserted within the receptacle 13 in the well 15 such that a set of baseline measurements may be recorded. Accordingly, the analogue output from the electrodes 7 is amplified 31, filtered 33 and stored 45 utilizing the equipment and methodology set out above. Throughout this baseline assessment stage and subsequently during the analysis of a compound, it is desirable to maintain the cell culture conditions substantially constant. Thus, the environment is monitored utilizing sensors deployed in the cell culture chamber 17 encompassing the MEA. The sensor output is monitored by a software module 18 running on the PC-based data acquisition system 23 or it may be monitored independently. In either case, the signals received form the sensor are employed to adjust environmental controls. The parameters which may be monitored could include temperature, pH and dissolved oxygen concentration of the culture medium.

The recording process for the baseline measurements is performed for each data channel corresponding to an electrode 7. For a given record (typically 100 seconds long) a set of so-called healthy channels is identified as follows by identifying the set of channels with the most frequently occurring non-zero number of spikes.

A compound (known or unknown) to be analyzed is then introduced to the cellular network covering the MEA 3. This may be applied directly to the network on the MEA 3 used for baseline assessment or a further equivalent sample of cultured network may be prepared on a further MEA 3 and inserted in the receptacle 13 in the well 15 in its place. Again, for a given record (typically 100 seconds in duration although a longer or shorter period may be selected) a set of so-called healthy channels is selected by identifying the set of channels with the most frequently occurring non-zero number of spikes. The healthy channels in each set i.e., the baseline measurements and the subsequent measurements in the presence of the compound are then compared further to identify the channels which are common to both sets. These common channels are then subjected to feature extraction to form a feature set.

To be useful in the subsequent detection and classification stages, a feature must be readily extractable from the data and numerically quantifiable. Various processing algorithms are used to extract features meeting this requirement. As many such features as possible are included in the set to encompass as much of the information content of the data as possible. The presence of redundant features in the set is tolerated. Furthermore, by averaging as late as possible in the process, the sensitivity of the features is found to be enhanced. The significance of a measured feature can also be estimated by calculating the standard deviation of that measure over all the selected channels.

The feature set can then be viewed as a vector quantity, with dimensions equal to the number of features; each component representing the numerical change to the feature in question equal to the difference between the baseline and subsequent measurements. Detection and classification reduces to performing manipulations on the response vectors. The detection process has been described above. The classification process is achieved by the use of standard cluster analysis by which is to be understood those mathematical clustering and partitioning techniques which can be used to group cases on the basis of their similarity over a range of variables e.g. component. Many clustering algorithms are available; they differ with respect to the method used to measure similarities (or dissimilarities) and the points between which distances are measured. Thus, although clustering algorithms are objective, there is scope for subjectivity in the selection of an algorithm. The most common clustering algorithms are polythetic agglomerative, i.e. a series of increasingly larger clusters are formed by the fusion of smaller clusters based on more than one variable. This hierarchical approach is particularly suited to computer based analysis in view of the large data sets which are to be analyzed. However, a less computationally intensive and therefore more rapid approach is the non-hierarchical k means, or iterative relocation algorithm. Each case is initially placed in one of k dusters, cases are then moved between clusters if it minimizes the differences between cases within a cluster. Depending on the computational capability of the PC-acquisition system and subject to any requirement for real-time analysis one of the aforementioned processes may be adopted.

In addition to the embodiment set out above, further variants having different MEA 3 arrangements are contemplated, some of which are set out below:
(1) SINGLE WELL, MULTIPLE CHAMBER ENVIRONMENT MEASURES—has sensors incorporated into the chamber apparatus to include simultaneous measurement and control of the culture environment (controlled perfusion, temperature, pH sensors).
(2) SINGLE WELL, MULTIPLE CHAMBER ENVIRONMENT MEASURES, MULTIPLE CELL PHYSIOLOGY MEASURES—as in (1) with integrated sensors to enable measurement of other cell functions such as intracellular calcium levels, lactate production etc.
(3) MULTI-WELL, MULTI-CHANNEL SYSTEM—instead of a single well format, 96 wells are formed and data retrieved from multiple microelectrodes in each well.
(4) MULTI-WELL, MULTI-PARAMETER SYSTEM—combination of (1) and (3) producing a drug screening device. A completely controlled multi-well assay system capable of controlled delivery of drug and fully automated data capture and analysis.
(5) MULTI-WELL, MULTI-PARAMETER SYSTEM, MULTIPLE CELL PHYSIOLOGY MEASURES. Combination of (2) and (3) to allow integrated analysis of many cell functions in many different assays.

The sensor/software systems of the invention can be configured for the detection of any natural or synthetic compound (known or unknown). For example, the sensor/software systems of the invention can be configured for the detection of a toxin (e.g., domoic acid (Amnesic Shellfish Poisoning, also referred to herein as “ASP”), saxitoxin (Paralytic Shellfish Poison, also referred to herein as “PSP”), crab toxin, Botulinum toxin, and Tetrodotoxin), a cell (e.g., a bacterial cell such as Vibrio bacteria or a histidine-producing bacteria, a diagnostic agent, a biomarker, or any chemical compound. In one particular embodiment, the sensor/software systems of the invention are configured for the detection of nerve agents, such as cholinesterase inhibitors including organophosphates and carbamates. Examples of organophosphates include Echothiophate, Diisopropyl fluorophosphate, Cadusafos, Cyclosarin, Dichlorvos, Dimethoate, Metrifonate (irreversible), Sarin, Soman, Tabun, VX, VE, VG, VM, Diazinon, Malathion and Parathion. Examples of carbamates include Aldicarb, Bendiocarb, Bufencarb, Carbaryl, Carbendazim, Carbetamide, Carbofuran, Carbosulfan, Chlorbufam, Chloropropham, Ethiofencarb, Formetanate, Methiocarb, Methomyl, Oxamyl, Phenmedipham, Pinmicarb, Pirimicarb, Propamocarb, Propham and Propoxur.

The sensor/software systems of the invention can also be configured for the detection of an environmental stimulus, such as change (e.g., increase or decrease) in atmospheric pressure and/or temperature, and/or a change (e.g., increase or decrease) in O₂, NO, N₂, NO₂, NO₃, CO, and/or CO₂ levels.

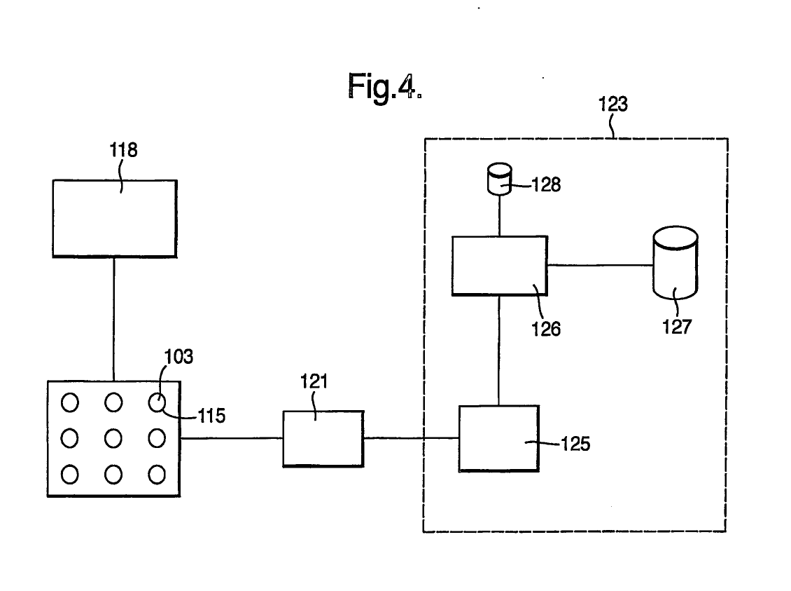

In an exemplary embodiment, FIG. 4 shows a schematic diagram of the sensor in which the cellular network is provided by a culture of cells derived from a species that is subject to a threat or a species whose response to a toxin or chemical compound (e.g., nerve agent), for example, may be extrapolated to another species such as humans. For example, the cellular network may comprise scallop heart cells. However, any cardio-myocyte like cells may be used, as previously described. The sensor 101 includes a plurality of chambers 117 each capable of housing an MEA 103. A perfusion system 118 permits the delivery of samples to be tested, for example a test sample derived from river water, to each MEA 103. The MEA 103 is as previously described in relation to the first embodiment in that it has a plurality of electrodes 107 arranged to contact the cellular network, in use. Electrical signals output from the electrodes 107 pass via an interconnect 119 to the amplifier unit 121 where the output is amplified. The amplifier unit 121 is a multichannel device capable of providing a gain of around 1000 to each channel. Typically, sufficient channels are available to allow each electrode 107 of the MEA 103 to be mapped to a channel. Depending on the configuration of the MEA 103 there may be need for more or less channels for satisfactory data collection. The amplifier unit itself is interfaced to a PC-based data acquisition system 123. The PC system 123 incorporates an Analogue to Digital conversion card 125 coupled via PCI bus to a central processor unit 126. The card 125 provides the external connections necessary to interface the analogue output of the amplifier 121 to the acquisition system 123. The card 125 is capable of sampling the amplified channel data from the amplifier unit 121 at up to 50 kHz/channel. The actual rate is determined by reference to the nature of cellular network and the resolution required to identify features of interest The central processor unit 126 carries out instructions provided by software and/or firmware necessary to analyze the digital data. The data may be analyzed in real time as events occur on the MEA 103 or retrieved from a storage device 127 such as a hard disk. In the former case, the storage device 127 may still be utilized to archive the data for later repeated or further analysis. The ability to proceed with real time as opposed to or off-line analysis will in some part depend on the rate at which data is generated and the storage capacity of the system 123.

The software and/or firmware includes a library 128 of feature sets corresponding to experimental or otherwise derived responses to a particular compound/stimulus (e.g., toxin(s), nerve agent(s)), to which the particular cellular network has been exposed. As such, the sensor 101 typically is required to perform an identification and preferably classification function in relation to a pre-defined set of feature sets, i.e. there is no requirement for the sensor 101 to identify every compound that is present in the sample only those whose effect might be toxic to the species under threat. Depending on the particular species under investigation, different libraries of feature sets may be loaded into the sensor 101. Conveniently, the sensor 101 is provided with software specific to the species with which a user is intending to work. Such software will contain the libraries as integrated portions of the software or as user loadable software modules. For example, accumulation of toxins in shellfish represents a growing problem.

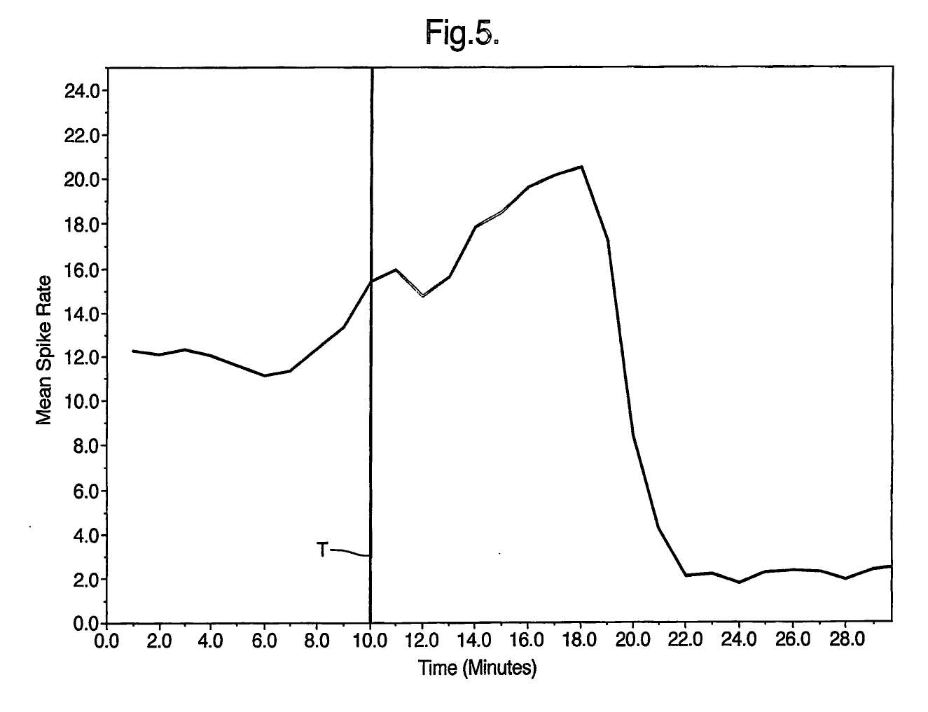

Two toxins are of particular importance because of their profound effects on the human nervous system. These are amnesiac shellfish poisoning (ASP) and paralytic shellfish poisoning (PSP) toxins. Ingestion of large doses of these toxins can result in death. Taking ASP poisoning as an example of the use of the sensor 101, it is known that the toxin primarily involved in ASP is domoic acid. Accordingly, before the sensor 101 may be utilized to detect the presence or otherwise of a particular toxin, it is necessary to provide a feature set for inclusion in the library 128 for subsequent use in the sensor. The creation of such a feature set or vector can be carried out using the system, described in the first embodiment. In the case of ASP, as has been indicated, the primary toxin is domoic acid. Thus, a network of cortical neuron cells from a mouse is applied to an MEA. The MEA is then placed within the well 115 and as has been described previously, the electrical response of the network to the addition of the compound, in this case domoic acid is extracted by the electrodes 107, amplified and the active channels identified and the relevant data captured. This data is then analyzed, again as has been described above and in the case of domoic acid it is found that one particularly useful feature indicative of domoic acid is Mean Spike Rate (MSR). FIG. 5 is a graph illustrating the mean spike rate from rat cortical neuron culture when exposed to domoic acid of 100 nM concentration at 10 minutes from the start of the data as indicated by the heavy line T. This response is then parameterized and stored in a file for inclusion in a software library of features.

Subsequently, a sensor 101 is used to determine the presence or otherwise of ASP in a sample of shellfish. The sensor 101 incorporates storage for a library of feature sets or vectors indicative of the responses to particular toxins realized by particular species. The library may be downloaded to the sensor such that it is held on a hard disk or other storage media 127 or it may be accessed via a network connection to a database. In one particular variant, the library is stored in an integrated circuit formed on the MEA 103 itself. Thus, by means of color coding or another indicia, an appropriate MEA 103 is selected having the requisite prestored library of feature sets or vectors applicable to a particular species under study. The integrated circuit is provided with the appropriate connections to the card edge connector to allow the library to be accessed by the sensor when installed in the receptacle in the well 115.

Either before or after installation in the receptacle 113, a cellular network of rat derived cortical neuron cellular material is deposited on an MEA 103. The MEA 103 is placed within the receptacle 113 in the well 115 of the sensor 101 and a shellfish sample is placed upon the MEA 103. The electrical response of the network to the addition of the compound (in this case the shellfish sample) is extracted by the electrodes 107, amplified and the active channels identified and the relevant data captured. This data is then analyzed against a library of feature sets including that obtained for ASP in relation to the MSR feature. If a match with a library response is found, within a predetermined limit of statistical confidence, then a positive result is flagged and an appropriate warning indication is given by the sensor 101 which may be visual, audio or a combination thereof. Where no such response is identified, again with a particular statistical confidence, then no such warning is given merely an indication with a level of confidence that the sample seems to be toxin free. Clearly, further features may exist which are found to correlate strongly with the presence or otherwise of a particular toxin. Such a feature set may be utilized in the analysis of unknown compounds and a match with one or more may be sufficient to cause the sensor to generate a warning indication.

In a further embodiment of the invention, the PC system 23 may be utilized as a measuring tool. Thus, the system 23 may be utilized to assess the physical and/or chemical characteristics of a known compound. For example, it has been recognized that the response of the system 23 to a particular compound may depend on the concentration of that compound. By establishing a library of features or feature set indicative of particular concentration levels at predetermined levels of statistical confidence, the concentration of a particular known compound may be identified. It will be further appreciated that the measurement tool may be integrated with the identification and classification function such that an initially unknown compound may be both identified and particularized in terms of its physical/chemical properties.

It will be recognized by those skilled in the art that a number of factors will impinge on the statistical confidence a user may have in the results of analysis using the method and apparatus of the invention. A quantification of the impact such factors might have may be incorporated in the statistical level of confidence applied to the results of a particular analysis. In order to minimize such effects, steps may be taken to utilize features that are normalized relative to a control. Alternatively, the minimum concentration to produce a significant response could be employed. Additionally, the response of the system may depend on the proximity of each cell to its nearest electrode. This may require the use of features that are independent of absolute amplitude, such as beat rate.

### Characterization of Biological Samples

The systems and methods provided by the invention are further useful for functionally characterizing living tissue (e.g., biopsied tissue), cells and organisms as a result of predictable responses to known compounds (e.g., for quality assurance/quality control of cells, tissue biopsies, and micro-organisms). The systems and methods provided by the invention can be used to characterize living tissue or cells from a specific origin, or to characterize an array of different living tissues or cells (from varying origins or selected for their sensitivity to specific compounds). Cells and tissues are currently characterized by morphology, simple biochemistry or biomarkers. These provide little information on the functional expression and integrity of the living tissue. Genomic analysis provides a fingerprint of the potential and epigenetics provide a measure of biochemical expression. However, such characterization methods are less sensitive than electrophysiological changes, which are predictably sensitive to small changes in the cell physiology and environment.

Any cardiomyocyte-like cells may characterized using the systems and methods described herein. As used herein, cardiomyocyte-like cells include cells that express an ion channel profile typical of a ventricular or atrial cardiomyocyte cell, cells that form a functional syncitium, and/or cells that beat (contract/expand, either spontaneously or in response to a chemical or electrical stimulus) in a rhythmic fashion. Examples of such cells include, but are not limited to cardiomyocytes and primary cells derived from heart tissue. Cardiomyocyte-like cells may be derived from any species that has a heart-like organ that physically pumps fluids around their body but typically refers to vertebrate species, particularly mammals (e.g., humans), birds, reptiles and fish, with a myogenic muscular organ that functions as part of a circulatory system to distribute blood around the body via rhythmic contraction and expansion. Examples of other suitable tissue or cells include, but are not limited to, stem cells (embryonic or non-embryonic), muscle cells, or neuronal cells, preferably of human origin.

Such cells are electrically active and this activity can be monitored using the systems and methods described above. A typical example might include the use of microelectrode array (MEA) technology, as described herein, to record the small, localized changes in voltage that occur when cultured cardiomyocyte-like cells expand and contract (‘beat’). These changes are measured via the use of small electrodes embedded into the plate, the signals of which are amplified and the response measured over time. Typically such a recording (typically in the form of a ‘waveform’) will be collected as an electronic file that can be read into analysis software. The waveforms are then mathematically described using the algorithms included in systems described herein.

For example, baseline ‘waveform’ data can be obtained from test tissue, cells, or organisms, and compared to a database of historic baseline or ‘reference’ responses of one or more types of cells. Baseline data should be collected under set, defined experimental conditions (e.g. including but not limited to a set recording duration, defined temperature, pH, gaseous conditions, culturing or test media/solution, standardized equipment, etc.). The electrical data (‘waveforms’) are then analyzed using the algorithms described herein to produce a mean or averaged waveform that represents a single ‘beat’ of activity from multiple recordings from that experiment—this may be the result of multiple simultaneous recordings (e.g. obtained from separate electrodes on the same plate), mathematically dissected beats or spikes from the same electrode over a short time period, or a series of separate recordings taken over a short time interval under identical experimental conditions (or a combination of the above). This waveform is then mathematically described and characterized via the algorithm(s) and compared to a historical reference waveform (or set of reference waveforms). In some embodiments, the reference set of waveforms are generated under specified experimental conditions and may represent the result of a summation of several recordings to generate an idealized reference recording. Differences between the test article and the reference waveform are then assessed. Acceptable levels of variance for each parameter individually and a summation of the changes from the reference waveform will be defined (e.g. a matrix of changes or ‘fingerprint’). Different parameters may be ‘weighted’ disproportionately in terms of the level of variance that is acceptable.

In certain aspects, this baseline ‘fingerprint’ may be indicative of an adverse event and/or effect associated with the test tissue, cell(s) or organism. For example, the baseline data may be indicative of prior exposure to one or more compounds such as a toxin or a nerve agent,

Waveform data can further generated in response to an external stimulus (e.g., a compound such as a drug, a diagnostic agent, a biomarker, toxin, nerve agent, or an environmental stimulus) applied to the test tissue, cell or organism. Data is generated in a similar fashion as described above. More specifically, the cells are exposed to an external, defined stimulus e.g. a set concentration of a reference drug or compound with a well defined effect upon the electrical activity of the cells. This might include (but is not limited to) chemicals known to interact with defined ion channel proteins present in cardiomyocyte-like cells e.g., an ion channel blocker. Once a set of recordings have been generated (e.g., as a baseline (‘control’) and drug-response (‘test’)), the algorithms described herein are used to analyze the differences between control and test to generate a set or matrix of changes (i.e., a ‘fingerprint’). This fingerprint can then be compared to a historical ‘reference’ drug or test response and differences between the QC test ‘fingerprint’ and the reference waveform ‘fingerprint’ will then be assessed. Acceptable levels of variance for each parameter individually and a summation of the changes from the reference waveform will be defined. Different parameters may be ‘weighted’ disproportionately in terms of the level of variance that is acceptable.

In certain embodiments, the characterization of cells and tissues based on their response to selected compounds using the systems/methods described herein enables the selection of appropriately personalized medicines having known effects on specific ion channels or other functional receptors such as G-protein coupled receptors Likewise, the characterization of cells and tissues based on their electrophysiological response to biomarkers or diagnostic agents can be used to optimize drug structure and function. The electrophysiological changes in response to biomarkers or diagnostic agents applied can further be used for diagnosing or localizing treatments.

In another embodiment, the knowledge of specific combinations of electrophysiological changes contained in each ‘fingerprint’ can be used to induce differentiation of cells or tissues or to activate specific pathways which might lead to provide natural protection (stimulate host organism natural pharmaceutical or ‘factors’), or to make implanted cells adaptable to the host environment (cell therapies), or to generate high yield of commercially valuable materials (cell culture etc).

In other certain embodiments, the detection and characterization of specific ion channel before or after exposure to an external stimulus, is useful for characterizing adverse events associated with pathology from biopsied cadaver tissue.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for characterizing functional activity of an isolated biological sample, said method comprising the steps of:
exposing the isolated biological sample to an external stimulus;
detecting a plurality of features indicative of physiological activity in response to the external stimulus using a sensor;
deriving a vector quantity based on the detected features, the vector quantity comprising a number of dimensions equal to a number of different features derived from the signal output of said sensor array;
comparing the derived vector quantity to a baseline of the physiological activity of the cell sample prior to exposure to the external stimulus to generate a physiological fingerprint of the cell sample; and
comparing the physiological fingerprint to a reference comprising a library of predetermined behavioral features of said biological sample, the comparison being indicative of one or more functional characteristics of the biological sample.

2. The method of claim 1, wherein the stimulus is a natural, a synthetic or an environmental stimulus.

3. The method of claim 2, wherein the natural stimulus is selected from the group consisting of a toxin, or a cell.

4. The method of claim 3, wherein the cell is a Vibrio bacteria or a histidine-producing bacterium.

5. The method of claim 3, wherein the toxin is crab toxin, saxitoxin, Botulinum toxin, Tetrodotoxin.

6. The method of claim 2, wherein the synthetic stimulus is selected from the group consisting of a diagnostic agent, a biomarker, or a chemical compound.

7. The method of claim 6, wherein the chemical compound is a cholinesterase inhibitor.

8. The method of claim 7, wherein the cholinesterase inhibitor is an organophosphate or a carbamate.

9. The method of claim 8, wherein the organophosphate is selected from the group consisting of: Echothiophate, Diisopropyl fluorophosphate, Cadusafos, Cyclosarin, Dichlorvos, Dimethoate, Metrifonate (irreversible), Sarin, Soman, Tabun, VX, VE, VG, VM, Diazinon, Malathion and Parathion.

10. The method of claim 8, wherein the carbamate is selected from the group consisting of: Aldicarb, Bendiocarb, Bufencarb, Carbaryl, Carbendazim, Carbetamide, Carbofuran, Carbosulfan, Chlorbufam, Chloropropham, Ethiofencarb, Formetanate, Methiocarb, Methomyl, Oxamyl, Phenmedipham, Pinmicarb, Pirimicarb, Propamocarb, Propham and Propoxur.

11. The method of claim 2, wherein the environmental stimulus is a change in atmospheric pressure, a change in temperature, a change in O₂ levels, or a change in CO₂ levels.

12. The method of claim 6, wherein the biomarker, diagnostic agent, or chemical compound is known.

13. The method of claim 1, wherein the biological sample comprises a tissue or a cell sample comprising one or more functional receptors or ion channels, or a combination thereof.

14. The method of claim 1, wherein the biological sample comprises an array of different tissues or a cell samples, each comprising one or more functional receptors or ion channels, or a combination thereof.

15. The method of claim 14, wherein the array of different tissues or cell samples are derived from varying origins, or are selected for sensitivity to one or more specific compounds.

16. The method of claim 1, wherein the biological sample comprises electrically active cells.

17. The method of claim 16, wherein the electrically active cells are primary cells derived from heart tissue, stem cells, cardiomyocytes, muscle cells, or neuronal cells.

18. The method of claim 17, wherein the stem cells are embryonic or non-embryonic stem cells.

19. The method of claim 1, wherein said physiological activity is static or changing physiological activity.

20. The method of claim 1, wherein the physiological activity is intracellular activity, extracellular activity, or a combination thereof.

21. The method of claim 1, wherein the physiological activity is electrical, chemical, fluorescent, or luminescent activity falling within the electromagnetic spectrum.

22. The method of claim 1, wherein the detected feature is an amplitude dependent feature.

23. The method of claim 1, wherein the detected feature is an electrical signal.

24. The method of claim 23, wherein the electrical signal is an intracellular signal.

25. The method of claim 23 wherein the electrical signal is generated by an external cellular membrane.

26. The method of claim 1, wherein the vector quantity is derived using a clustering algorithm selected from a polythetic agglomerative algorithm, a k-means algorithm or an iterative relocation algorithm.

27. The method of claim 1, wherein the sensor comprises a single electrode.

28. The method of claim 1, wherein the sensor is a sensor array comprising a plurality of electrodes.
